(19) United States
(12) Patent Application Publication
(10) Pub. No.: US 2022/0169979 A1
(43) Pub. Date: Jun. 2, 2022

(54) METHODS FOR ENRICHING POPULATIONS OF CELLS

(71) Applicants: Rutgers, The State University of New Jersey, New Brunswick, NJ (US); Life Science Institute, Inc., Tokyo (JP)

(72) Inventors: Wise Young, New Brunswick, NJ (US); Dongming Sun, Princeton Junction, NJ (US); Iman Tadmori, Berkeley Heights, NJ (US); Zikuan Leng, Zhengzhou (CN); Mari Dezawa, Sendai (JP)

(73) Assignees: Rutgers, The State University of New Jersey, New Brunswick, NJ (US); Life Science Institute, Inc., Tokyo (JP)

(21) Appl. No.: 17/602,557

(22) PCT Filed: Apr. 9, 2020
(86) PCT No.: PCT/US20/27375

§ 371 Date: Oct. 8, 2021

Related U.S. Application Data
(60) Provisional application No. 62/831,491, filed on Apr. 9, 2019.

(51) Int. Cl. C12N 5/073; A61K 35/51; A61K 35/28

(52) U.S. Cl. CPC C12N 5/0605; A61K 35/51; A61K 35/28; C12N 2502/45; C12N 2502/1114; C12N 2502/1164; C12N 2501/115

(57) ABSTRACT

This disclosure describes efficient methods for separating desired populations of cells, including Multilineage-Differentiating Stress-Enduring (MUSE) cells. Also described are the methods for isolating and enriching MUSE cells through a sorting, expanding, and re-sorting procedure. The enriched cells or cell populations can be used for treating cancer, repairing various tissues, and treating various degenerative or inherited diseases.

### CROSS-REFERENCE TO RELATED APPLICATIONS

This application claims priority under 35 U.S.C. § 119(e) to U.S. Provisional Patent Application No. 62/831,491, filed Apr. 9, 2019. The foregoing application is incorporated by reference herein.

### FIELD OF THE INVENTION

This invention relates generally to methods for enriching desired populations of cells and more specifically to methods for enriching desired populations of cells including Multilineage-Differentiating Stress-Enduring (MUSE) cells and uses thereof.

### BACKGROUND OF THE INVENTION

Multilineage-Differentiating Stress-Enduring (MUSE) cells are a subtype of mesenchymal stem cells (MSCs) that express the state-specific embryonic antigen 3 (SSEA3). MUSE cells can differentiate into endodermal-, ectodermal- and mesodermal-lineage cells spontaneously in vitro or can be induced to produce cell types from all three lineages. They can self-renew but do not form teratomas in vivo. MUSE cells migrate to tissues that express sphingosine-1, integrate into damaged tissues in vivo when administered intravenously, differentiate into specific cells needed to repair tissues, and survive over six months in animals. MUSE cells stimulate tissue regeneration and restore functions in many animal disease models, e.g., liver diseases, stroke, muscle regeneration, skin regeneration, malignant gliomas, and myocardial infarction. After in vivo transplants in animals, no tumors have been reported. MUSE cells also have low telomerase activity and low expression of cell-cycle genes compared to embryonic stem (ES) and induced pluripotent stem (iPS) cells.

MUSE cells pose several advantages over other stem cells for regenerative medicine. First, they are pluripotent adult stem cells that can produce themselves and many other types of cells to repair a wide variety of tissues. Second, MUSE cells have been isolated from many tissues and available from autologous and allogeneic sources, including fat, bone marrow, adult blood, umbilical cord blood, and umbilical cord. Third, MUSE cells can be identified by a combination of SSEA3 and a mesenchymal marker such as CD105, CD29, and CD90. Because mesenchymal cells attach to and grow well on plastic, nearly 100% of cells cultured on plastic from Wharton's Jelly (WJ) or Cord Lining (CL) express mesenchymal markers. Culturing the cells on plastic effectively purifies the cells to be a mesenchymal population. It was found that MUSE cells can be sorted and counted from cell cultures grown on plastic based on SSEA3 expression alone. Finally, unlike other pluripotent cells such as embryonic or induced pluripotent stem (iPS) cells, MUSE cells do not form teratomas or other tumors. When grown in culture, their self-renewal rates are slower than their production of non-MUSE differentiated cells, and therefore the percentage of MUSE cells invariably decline over time in cultures.

SSEA3+ cells make up of 0.03% to several percent of mesenchymal cells cultured from goatskin, human dermal fibroblasts, adipose tissue, and bone marrow. To isolate MUSE cells, fluorescence-activated cell sorting (FACS) is commonly used, but this method is inefficient and expensive (Heneidi, S., et al. PLoS One, 2013. 8(6): p. e64752). Some other methods include the use of enzymes or applying stress to the cells, relying on the stress-resistance of MUSE cells to survive while other cells die. In populations of mesenchymal cells purified by these methods, only 11.6% could form MUSE cell clusters (Kuroda, Y., et al. PNAS, 2010. 107(19): p. 8639-43; Dezawa, M., Cell Transplant, 2016. 25(5): p. 849-61).

Thus, there remains a strong need for efficient methods for obtaining high purity and high yield of cells, such as MUSE cells.

### SUMMARY OF INVENTION

This disclosure addresses the need mentioned above in a number of aspects. In one aspect, this disclosure provides a method for enriching MUSE cells. The method comprises: (i) providing a cell or tissue source of MUSE cells; (ii) separating a first population of cells from the cell or tissue source of MUSE cells, wherein the first population of cells is separated by selecting for SSEA3+ cells and comprises SSEA3+ MUSE cells; (iii) culturing at least a sub-population of the first population of cells in a culture medium; (iv) repeating step (iii) at least 1-10 passages; and (v) separating from resulting cultured cells a population of enriched MUSE cells by selecting for SSEA3+ cells, whereby the population of enriched MUSE cells comprises about or greater than 80% of SSEA3+ MUSE cells. In some embodiments, the culture medium comprises basic fibroblast growth factor (bFGF).

In some embodiments, the method further comprises: separating from the cell or tissue source of MUSE cells a second population of cells and a third population of cells, wherein the second population of cells is separated by selecting for CD4+ and CD8+ cells before or after the first population of cells are separated from the cell or tissue source of MUSE cells, and the third population of cells is recovered after the first population of cells and the second population of cells are separated from the cell or tissue source of MUSE cells. In some embodiments, the second population of cells comprises T- and natural killer (NK) lymphocytes. In some embodiments, the third population of cells comprises CD14+ monocytes, CD34+ endothelial progenitor cells, or CD133+ pluripotent cells.

In some embodiments, the cell or tissue source of MUSE cells is obtained from a tissue of an animal, such as umbilical cord blood, umbilical cord, umbilical cord stroma cells (Wharton's jelly), amniotic membranes, placenta, umbilical cord lining, menstrual blood, peripheral blood, bone marrow, skin, or adipose. In some embodiments, the animal is a mammal (e.g., human). In some embodiments, the cell or tissue source of MUSE cells comprises mesenchymal cells or mononuclear cells.

In some embodiments, the first population of cells is separated using an immunoaffinity-based reagent comprising an SSEA3 antibody. In some embodiments, the second population of cells is separated using an immunoaffinity-based reagent comprising CD4 and CD8 antibodies.

In some embodiments, the SSEA3 antibody or the CD4 and CD8 antibodies are monoclonal antibodies, such as a mouse monoclonal IgG or IgM antibodies or a rat monoclonal IgG or IgM antibodies. In some embodiments, the SSEA3 antibody or the CD4 and CD8 antibodies are conjugated to magnetic particles.

Also within the scope of this disclosure is a pharmaceutical composition comprising the MUSE cells enriched by the method as described above.

In another aspect, this disclosure provides a cell therapy composition for allotransplantation comprising the MUSE cells enriched by the method as described above.

In yet another aspect, this disclosure provides a method for regenerating tissue in a subject (e.g., human). The method comprises administering to the subject an effective amount of the MUSE cells enriched by the method as described above.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A and 1B (collectively “FIG. 1”) are flowcharts showing exemplary methods for enriching MUSE cells.

FIG. 2 is a flowchart showing an exemplary method for enriching desired populations of cells.

FIG. 3 shows an apparatus for implementing a disclosed method for enriching MUSE cells. The apparatus comprises two containers (container 1 and container 2) with two spouts each. Spout B is connected by tubing to spout B on another container, so that liquid content can be poured from container 1 to container 2. The containers may include magnets (cut-away) surrounding each of the containers to attract cells attached to antibody-coated magnetic microbeads.

FIG. 4 shows a linear relationship between the number of WJ-MSC cells and cord weight.

FIGS. 5A and 5B (collectively “FIG. 5”) show expression levels of CD105+ and SSEA3+/CD105+ in Cord Lining (CL) cells (FIG. 5A) and Wharton's Jelly (WJ) cells (FIG. 5B). Gray bars indicate the percent of cells expressing CD105+. Black bars indicate the percent of cells expressing both CD105 and SSEA3. The numbers refer to different samples. P0, P1, and P2 indicate passages 1, 2, and 3. One-way ANOVA shows SSEA3+ percentages dropped sharply between P0 and P1 in the CL group and P0 and P2 in the WJ Group.

FIG. 6A shows a phase-contrast image of Wharton's Jelly cells (10×). FIG. 6B shows a phase-contrast image of Cord Lining cells (10×). The tissue was seeded in the dashed circle, where the MSCs started to grow. The scale bar indicates 100 μm.

FIG. 7 shows flow cytometry analysis of HUC derived MSCs. A sample was taken from 96WJ P2. SSC-A and FSC-A were used to gate cells out of the debris, and FSC-H and FSC-A were used to gate single cells other than clusters. Propidium iodide (PI) staining was used to exclude the dead cells. The results show that 92.98% of the total cells were alive, 1.55% were SSEA3+, and over 99% were CD105+, CD90+, CD73+, CD44+, CD166+, and CD29+. The cells were CD14− and CD45−.

FIG. 8 shows flow cytometry results of post-magnetically activated cell sorting (post-MACS) isolated SSEA3+ cells from 96WJ P2. A sample was analyzed right after sorting. About 89.84% of the total cells were alive. 92.31% of the sorted population was SSEA3+, while the other 7.27% seemed to be debris according to the diameter. Over 99.5% were CD105+, CD29+, CD90+, CD73+, CD44+, and CD166+. The cells were CD14− and CD45−.

FIG. 9 shows percentages change of SSEA3+ cells after the MACS in the following ten passages. 96WJ-P2-MACS-P0 was right after the magnetic sorting, and 93.77% of the total population were SSEA3+ but CD14-. The other 6.19% were debris according to the diameter. The sorted cells were cultured, and the next passage cells were collected every four days. In the first passage, the SSEA3+ percentage decreased to 14.8%, but SSEA3+ percentages ranged from 62.5% to 75.9% between P2 to P5. The percentages of SSEA3+ cells declined to 42.0%-54.7% between P6 to P9. Even in P10, the cultures still contained 37.3% SSEA3+ cells. After P10, the cells were re-sorted, and an 89.4% SSEA3+ culture was achieved. In all passages, the CD105+ percentages remained over 99.0%.

FIGS. 10A, 10B, and 10C show staining of 96CL Passage 1. FIG. 10A shows Hoechst staining of 96CL Passage 1. FIG. 10B shows Ki-67 staining of 96CL Passage 1. FIG. 10C shows a merged image of FIG. 10A and FIG. 10B. Antigen Ki-67 is a nuclear protein that is a marker of proliferating cells. About 65% of the total cells were Ki-67+ indicating that the proliferation of the population was very active. Scale bar=50 μm.

FIG. 11 shows doubling time (TD) of MUSE and Non-MUSE cells at 10 passages after MACS. The left axis indicates hours for cell doubling of MUSE cells and Non-MUSE cells. The right axis indicates the percent of MUSE cells. The TD of MUSE cells in P1 was 403 hours indicating they nearly did not proliferate, while it was 14.4 hours for Non-MUSE cells. From P2 to P7, the TD of MUSE cells was 24.9±5.4 hours indicating that the number of MUSE cells doubled about once a day, while from P8 to P11, TD increased to 39.8±5.4 hours. As for Non-MUSE cells, the TD was rather stable at 31.2±7.8 hours from P2 to P11. The data suggest that P2 to P7 were the best passages to re-sort in order to achieve millions of MUSE cells.

FIG. 12 shows the levels of microbead with an SSEA3 antibody attaching to sorted cells after the MACS. Of the sorted SSEA3+ cells (96.29%), 99.19% had labelled microbeads, and the signal was strong. These results indicate that the microbeads worked very well.

### DETAILED DESCRIPTION OF THE INVENTION

This disclosure describes a method to isolate and enrich large numbers of healthy MUSE cells efficiently and inexpensively, for example, from mesenchymal cells isolated from human umbilical cord (HUC). In one example, the method employs magnetically activated cell sorting (MACS) to isolate SSEA3+ cells, followed by cell expansion in culture, and then a second MACS procedure to obtain a high purity cell population with about or greater than 80% SSEA3+ cells.

The disclosed method is advantageous in several aspects. First, the method is gentle and does not damage the cells. The anti-SSEA3 antibody-coated beads bind to SSEA3 on the surface of cells and move the cells towards magnets applied to the container walls, allowing non-SSEA3 expressing cells to pass through. Second, the method is highly efficient, allowing billions of cells to be sorted in a matter of minutes. Third, the method preserves non-MUSE cells, allowing them to flow through, be analyzed or sorted again, which can also be used as control cells for comparison with the MUSE treatment. Fourth, the isolated MUSE cells should have little or no regulatory barrier since MACS-sorted cells have long been used in clinical trials of CD34+ cells (Richel, D. J., et al. Bone Marrow Transplant, 2000. 25(3): p. 243-9). Finally, the method yields relatively high purity of SSEA3+ cells (e.g., >80% SSEA3+ cells), superior to previously published studies using MACS which yielded 77.1% and 71.3% isolated MUSE cells (Uchida, H., et al. Stroke, 2017. 48(2): p. 428-435; Kinoshita, K., et al. Stem Cells Transl Med, 2015. 4(2): p. 146-55).

This disclosure also describes an efficient method to separate from starting cells (e.g., mononuclear cells) desired populations of cells, such as T- and NK-lymphocytes, SSEA3+ MUSE cells, and CD14+ monocytes with CD34+ endothelial precursors and CD133+ pluripotent stem cells. The lymphocytes can be selected with CD4 and CD8 antibody-coated microbeads. They can be modified to express chimeric antigen receptors (CAR) to produce CAR-T and CAR-NK cells to target specific tumors. The MUSE cells can be selected with SSEA3 antibody-coated microbeads and expanded in adherent cultures to produce a large number of pluripotent MUSE cells. MUSE cells can be used to repair liver, lung, heart, kidney, brain, and other tissues. The remaining cells are enriched for CD14+ monocytes, CD34+ endothelial progenitor cells, and/or CD133+ pluripotent cells, which are believed to be a source of M2 macrophages that secrete growth factors to regenerate the spinal cord and brain. These three populations of cells can be administered in different proportions, depending on the clinical condition and timing.

### I. METHODS FOR ENRICHING DESIRED POPULATIONS OF CELLS

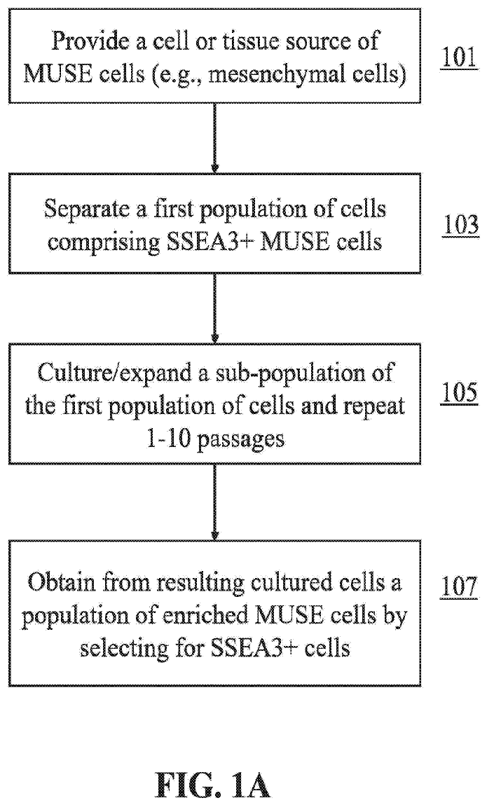

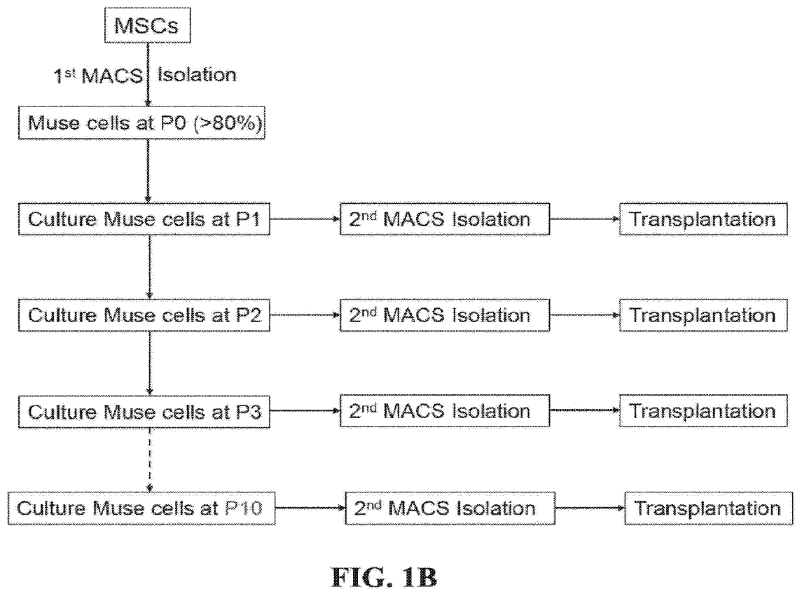

FIG. 1A shows a method for enriching MUSE cells. The method includes: (i) providing a cell or tissue source of MUSE cells at 101; (ii) at 103, separating a first population of cells from the cell or tissue source of MUSE cells, wherein the first population of cells is separated by selecting for SSEA3+ cells and comprises SSEA3+ MUSE cells; (iii) culturing at least a sub-population of the first population of cells in a culture medium at 105; and (iv) repeating step (iii) at least 1-10 passages (e.g., at least 1 passage, at least 2 passages, at least 3 passages, at least 4 passages, at least 5 passages, at least 6 passages, at least 7 passages, at least 8 passages, at least 9 passages, at least 10 passages). At 107, the method further includes separating from resulting cultured cells a population of enriched MUSE cells by selecting for SSEA3+ cells, whereby the population of enriched MUSE cells comprises about or greater than 80% of SSEA3+ MUSE cells. In some embodiments, the cell or tissue source of MUSE cells comprises mesenchymal cells or mononuclear cells.

FIG. 1B shows an example of a process for enriching MUSE cells. First, the cell or tissue source of MUSE cells is subject to MACS isolation, for example, by selecting for SSEA3+ cells. Second, a sub-population of the isolated MUSE cells can be cultured at least for 1 to 10 passages. The resulting cultured cells are then subject to a second MACS isolation to enrich MUSE cells, for example, by selecting for SSEA3+ cells. The enriched MUSE cells can be used for a variety of applications, including transplantation. Uses of the enriched MUSE cells are further described in the latter sections of this disclosure.

The term “culturing” refers to maintaining cells under conditions in which they can proliferate and avoid senescence. For example, cells are cultured in media optionally containing one or more growth factors, i.e., a growth factor cocktail.

The term “expansion” refers to the cultivation of cells in vitro. Such cells can be extracted from a mammal and additional quantities of cells generated by cultivation in the appropriate environment, e.g., in media containing a growth factor. If possible, stable cell lines are established to allow for continued propagation of cells.

The culture medium for culturing/expanding cells can be a basal medium, e.g., DMEM/F-12 (GIBCO), used for supporting the growth of cells. The culture medium may include basic fibroblast growth factor (bFGF). In some embodiments, the culture medium may include about 0.1 ng/mL to 100 ng/mL bFGF (e.g., 0.5 ng/mL, 1 ng/mL, 2 ng/mL, 5 ng/mL, 10 ng/mL, 20 ng/mL, 50 ng/mL). In some embodiments, the culture medium may include about 1% to about 20% FBS, about 0.5 mM to about 10 mM GlutaMAX™-I, about 0.1% to about 5% PSA, and about 0.1 ng/mL to about 100 ng/mL bFGF. In some embodiments, the culture medium is a DMEM/F-12 basal medium including about 1% to about 20% FBS, about 0.5 mM to about 10 mM GlutaMAX™-I, about 0.1% to about 5% PSA, and about 0.1 ng/mL to about 100 ng/mL bFGF. In some embodiments, the culture medium is a DMEM/F-12 basal medium including about 10% FBS, about 2 mM GlutaMAX™-I, about 1% PSA, and about 1 ng/mL bFGF.

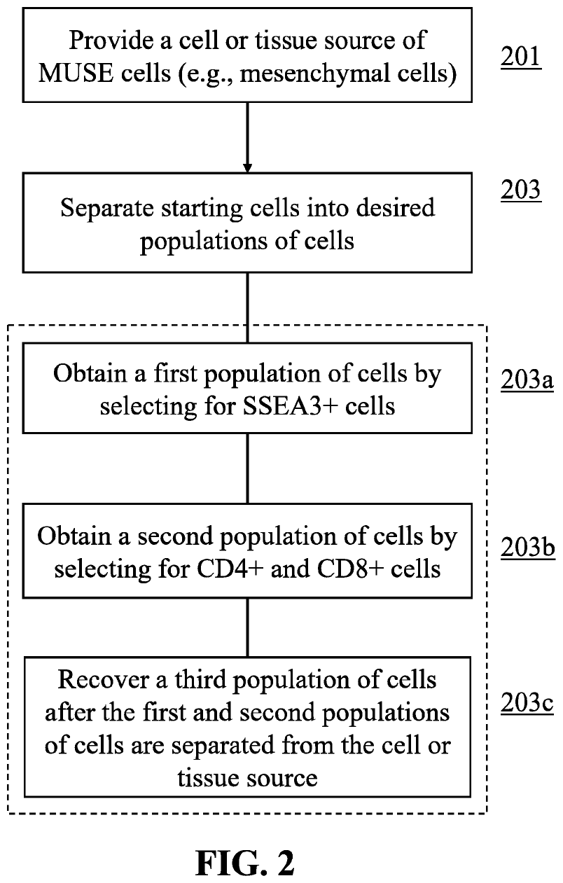

FIG. 2 shows a method for enriching desired populations of cells. The method includes: (i) providing a cell or tissue source of MUSE cells at 201; and (ii) at 203, separating from the starting cells a first population of cells, a second population of cells, and a third population of cells. At 203a, the first population of cells is obtained by selecting for SSEA3+ cells. At 203b, the second population of cells is obtained by selecting for CD4+ and CD8+ cells. At 203c, the third population of cells is recovered after the first population of cells and the second population of cells are separated from the cell or tissue source of MUSE cells.

In some embodiments, the second population of cells comprises T- and natural killer (NK) lymphocytes. In some embodiments, the third population of cells comprises CD14+ monocytes, CD34+ endothelial progenitor cells, or CD133+ pluripotent cells.

Isolating the first population of cells and isolating the second population of cells can be performed in any order. In one example, isolating the first population of cells is performed prior to isolating the second population of cells. In another example, isolating the first population of cells is performed after isolating the second population of cells.

In some embodiments, the first population of cells is separated using an immunoaffinity-based reagent comprising an SSEA3 antibody. In some embodiments, the second population of cells is separated using an immunoaffinity-based reagent comprising CD4 and CD8 antibodies.

In some embodiments, the SSEA3 antibody or the CD4 and CD8 antibodies are monoclonal antibodies, such as a mouse monoclonal IgG or IgM antibodies or a rat monoclonal IgG or IgM antibodies. In some embodiments, the SSEA3 antibody or the CD4 and CD8 antibodies are conjugated to magnetic particles.

Non-limiting examples of the CD4 antibody may include 4B12 (THERMO FISHER SCIENTIFIC), NBP1-19371 (NOVUS BIOLOGICALS), MAB3791 (R&D SYSTEMS), and MT310 (SANTA CRUZ BIOTECHNOLOGY).

Non-limiting examples of the CD8 antibody may include YTS169.4 rat anti-mouse CD8 antibody (BIO-RAD), mouse anti-rat CD8 antibody (NOVUS BIOLOGICALS), anti-murine CD8a antibody (DIANOVA), Goat anti-Feline CD8 polyclonal antibody (NOVUS), and others. Anti-human CD8 antibodies are likewise available, i.e., mouse anti-human CD8 Antibody Clone RAVB3 (BIOSOURCE), ab4055 and ab203035 (ABCAM), YTS169.4 (BIO-RAD), MAB1509 (R&D SYSTEMS), and 32-M4 (SANTA CRUZ BIOTECHNOLOGY).

Non-limiting examples of the SSEA3 antibody may include MA1-020 and MC-631 (THERMO FISHER SCIENTIFIC), LS-C179938 (LSBio), and 15B11 (IBL).

In some situations, it is useful to isolate monocytes (e.g., CD14+ monocytes). Monocytes are precursors to M1 and M2 macrophages, important for cleaning up and stimulating repair of damaged tissues. Monocytes can also differentiate into dendritic cells that play a role in antigen presentation to activate the immune system. CD14 antibodies are often used to isolate monocytes. CD14 binds lipopolysaccharide (LBS) in the presence of lipopolysaccharide-binding protein (LPB), but it also recognizes other pathogen-associated molecules such as lipoteichoic acid. Commercially available CD14 antibodies include, but are not limited to, UCHM-1 (MILLIPORESIGMA ANTIBODIES), anti-CD14 antibodies (SINO BIOLOGICAL), 5A3B11B5 CD14 antibody (SANTA CRUZ BIOTECHNOLOGY), 4B4F12 anti-CD14 antibody (ABCAM), Clone M5E2 (STEMCELL TECHNOLOGIES), HCD14 CD14 antibody (BIOLEGEND), Invitrogen CD14 antibody (EBIOSCIENCES), human CD14 antibody MAB3832-100 (R&D SYSTEMS), Clone TüK4 (BIO-RAD), etc.

The term “antibody” (Ab) as used herein includes monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE, and IgM); and biologically relevant fragment or specific binding member thereof, including but not limited to Fab, F(ab′)2, Fv, and scFv (single chain or related entity). It is understood in the art that an antibody is a glycoprotein having at least two heavy (H) chains and two light (L) chains inter-connected by disulfide bonds, or an antigen-binding portion thereof. Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

The term “monoclonal antibody” as used herein refers to an antibody obtained from a population of substantially homogeneous antibodies, i.e., the individual antibodies comprising the population are identical except for possible naturally occurring mutations that may be present in minor amounts. The term “polyclonal antibody” refers to preparations that include different antibodies directed against different determinants (“epitopes”).

In some embodiments, the cells isolated and/or enriched by the disclosed methods are substantially pure. The term “substantially pure” means that the specified cells constitute a substantial portion of or the majority of cells in the preparation (i.e., more than 20%, 30%, 40%, 50%, 60%, 70%, 80%, 90%, or 95%). Generally, a substantially purified population of cells constitutes at least about 70% of the cells in a preparation, usually about 80% of the cells in a preparation, and particularly at least about 90% of the cells in a preparation (e.g., 95%, 97%, 99% or 100%).

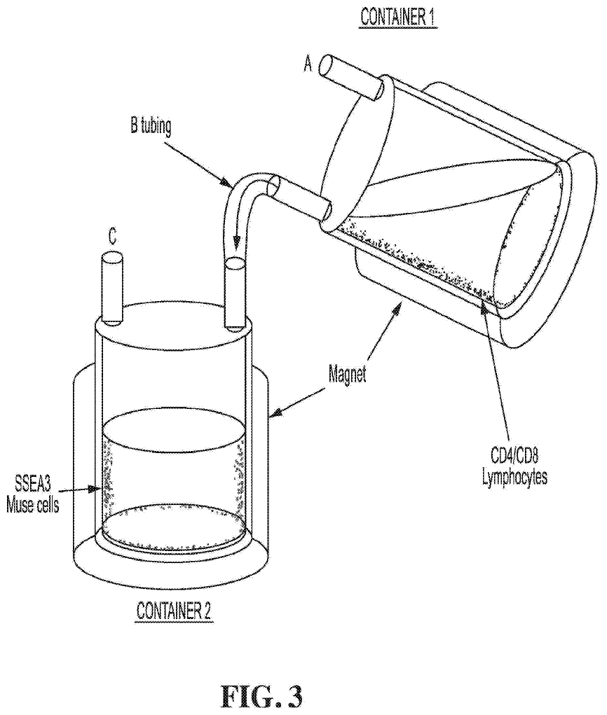

FIG. 3 shows an apparatus for implementing the disclosed methods. The apparatus enables two sequential MACS sorts to isolate three populations of cells from various sources, such as umbilical cord blood-derived mononuclear cells (UCBMNC). The apparatus includes two containers (e.g., container 1 and container 2) with two spouts each. Spout B is connected by tubing to a second spout B on another container. Through the tubing connecting the two spouts B, liquid content can be poured from one container to the other container. The apparatus also includes magnets (cut-away) surrounding each of the containers to attract cells attached to antibody-coated magnetic microbeads. The antibody-coated microbeads are retained in the containers when the magnets are present. The cell suspension is injected through spout A and poured through spout B. The remaining cell suspension can be poured out through spout C. The separate populations of cells can be washed directly in the two containers and collected from the containers through spouts A and C, after the reusable magnets are removed. In one example, container 1 may include CD4 and CD8 antibodies-coated magnetic microbeads for selecting CD4/CD8 lymphocytes, and container 2 may include SSEA3 antibody-coated magnetic microbeads for selecting SSEA3+ MUSE cells. In another example, container 1 may include SSEA3 antibody-coated magnetic microbeads for selecting SSEA3+ MUSE cells, and container 2 may include CD4 and CD8 antibodies-coated magnetic microbeads for selecting CD4/CD8 lymphocytes.

As used herein, the term “MUSE cells” refers to the pluripotent stem cells described in Kuroda et al., 2010 and Wakao et al., 2011, as well as US Patent Application Nos. 20120244129 and 20110070647, the contents of which are incorporated herein by reference in their entireties. More specifically, MUSE cells refer to a specific type of animal (e.g., human) mesenchymal pluripotent stem cell that is capable of generating cells with the characteristics of all three germ layers from a single cell. MUSE cells are stress tolerant; morphologically indistinguishable from general mesenchymal cells in adhesion culture (resembling fibroblasts); able to form M-clusters in suspension culture that is positive for pluripotency markers and alkaline phosphatase staining; able to self-renew; not very high in their proliferation activity and not shown to form teratomas in immunodeficient mouse testes; able to differentiate into endodermal, ectodermal, and mesodermal cells both in vitro and in vivo; and positive for both CD105 and SSEA3.

MUSE cells may also express pluripotency markers such as Nanog, Oct3/4, and Sox2, and are negative for NG2 (a marker for perivascular cells), CD34 (a marker for endothelial progenitors and adipose-derived stem cells), von Willebrand factor (a marker for endothelial progenitors), CD31 (a marker for endothelial progenitors), CD117 (c-kit, a marker for melanoblasts), CD146 (a marker for perivascular cells and adipose-derived stem cells), CD271 (a marker for neural crest-derived stem cells), Sox10 (a marker for neural crest-derived stem cells), Snail (a marker for skin-derived precursors), Slug (a marker for skin-derived precursors), Tyrp1 (a marker for melanoblasts), and Dct (a marker for melanoblasts) by flow cytometry analysis or by RT-PCR.

MUSE cells from bone marrow, fibroblast, or adipose tissue are limited in number and growth capacity. The cells are not abundant in bone marrow aspirates and about only 1:3,000 of bone marrow mononucleated cells are MUSE cells. In mesenchymal cell cultures, MUSE cells account for only several percentages of fibroblasts and bone marrow stromal cells. Once isolated and cultured in suspension, MUSE cells typically grow for only several weeks and then cease proliferation but, after transferring to adherent culture, they start proliferation. Accordingly, merely isolating CD105+/SSEA3+ cells from marrow mononuclear cells and subsequent conventional culturing such isolated cells may not provide sufficient MUSE cells for practical uses.

Even though MUSE cells have limited proliferation in suspension cultures, they keep on growing until their Hayflick limit in adherent culture. This limit is 40-60 divisions in human fetal cell cultures. In cultures of older adult cells, depending on the age of the cells, the Hayflick limit should be less. Umbilical cord blood cells, being the youngest post-natal source of cells, should have more proliferation capacity Similar to other somatic stem cells and hematopoietic stem cells. MUSE cells generate themselves by symmetric cell division but, at the same time, randomly produce non-MUSE cells by asymmetric cell division. Therefore, initially purified MUSE cell cultures show a sigmoidal decline in their concentration in culture, reaching a plateau of several percent, and then maintain this lower concentration. Yet, as disclosed herein, the method of this invention allows one to isolate and increase the concentration of MUSE cells in vitro.

The disclosed methods can be used to isolate, enrich, or expand MUSE cells from various tissues. In some embodiments, the starting cells are obtained from umbilical cord blood. Umbilical cord blood is an attractive source of MUSE cells for the following reasons. First, HLA-matched umbilical cord blood is a rich and immune-compatible source of MUSE cells. Many umbilical cord blood banks have stored hundreds of thousands of cord blood units that can be HLA-matched to provide immune-compatible MUSE stem cells for transplantation purposes. Second, umbilical cord blood cells have greater expansion potential than other sources of adult mesenchymal stem cells obtained from bone marrow, skin, or fat. Third, umbilical cord blood has a long history of safe use in bone marrow replacement with a low tumorigenesis risk.

The disclosed methods are also applicable for isolating, expanding, or enriching MUSE cells from other tissues besides umbilical cord blood. MUSE cells are a special subpopulation of pluripotent stem cells isolated from mesenchymal stem cells. Thus, any sources suitable for isolating mesenchymal stem cells can be used to practice the disclosed methods. Non-limiting examples of such sources include umbilical cord, umbilical cord stromal cells (Wharton's jelly), amniotic membranes, placenta, umbilical cord lining, and even menstrual blood. Other examples include bone marrow, skin, adipose tissues, and even peripheral blood. However, as pointed out above, none of these sources have as many MUSE cells and may have less growth potential than umbilical cord blood cells.

Once the desired populations of cells (e.g., MUSE cells) are isolated or enriched, the cells can then be tested by standard techniques to confirm the differentiation potential of the cells using one or more of lineage-specific markers. That is, one can test whether, under suitable culturing conditions, the cells can be induced to differentiate and give rise cells expressing markers for the three germ layers. Exemplary markers for ectodermal cells include nestin, NeuroD, Musashi, neurofilament, MAP-2, and melanocyte markers (such as tyrosinase, MITF, gf100, TRP-1, and DCT); exemplary markers for mesodermal cells include brachyury, Nkx2-5 smooth muscle actin, osteocalcin, oil red-(+) lipid droplets, and desmin; exemplary markers for endodermal cells include GALA-6, α-fetoprotein, cytokeratin-7, and albumin.

For example, isolated/enriched cells can be induced to form neuro-glial cells, osteocyte, and adipocyte by methods known in the art. Briefly, the cells can be passed and cultured to confluence, shifted to an osteogenic medium or an adipogenic medium and incubated for a suitable time (e.g., 3 weeks). The differentiation potential for osteogenesis can be assessed by the mineralization of calcium accumulation, which can be visualized by von Kossa staining. To examine adipogenic differentiation, intracellular lipid droplets can be stained by Oil Red 0 and observed under a microscope. For neural differentiation, the cells can be incubated in a neurogenic medium for a suitable duration (e.g., 7 days), and then subjected to serum depletion and incubation of β-mercaptoethanol. After differentiation, cells exhibit the morphology of retractile cell body with extended neurite-like structures arranged into a network. An immunocytochemical stain of lineage-specific markers can be further conducted to confirm neural differentiation. Examples of the markers include neuron-specific class III β-tubulin (Tuj-1), neurofilament, and GFAP.

### II. COMPOSITIONS AND METHODS OF TREATMENT

The three populations of cells enriched by the above-described methods have beneficial effects on many conditions and can be used in many ways. For example, Lymphocytes, such as T-cells and NK-cells are shown to be toxic to tumor cells when modified to express chimeric antigen receptors (CAR) to specific tumor antigens. Monocytes with CD34/CD133+ progenitor are macrophage precursor cells. Macrophages have three phenotypes: M1 is the pro-inflammatory phagocyte; M2 is the anti-inflammatory reparative phagocyte; and dendrocytes phagocytose dead or dying cells to present their antigens to immune cells. Monocytes are likely to be the effector cells that stimulate regeneration when transplanted into the spinal cord. CD34+/CD133+ cells are respectively endothelial precursors and pluripotent umbilical cord blood stem cells. Both may stimulate monocytes to produce M2 macrophages. But CD34+ cells are of interest because they participate in vasculogenesis and possibly hematopoiesis. CD133+ cells are pluripotent cells that may be useful for differentiating into many kinds of cells.

MUSE cells are pluripotent mesenchymal stem cells, capable of differentiating into the three germ layers through in vitro adherent culture. Specifically, the pluripotent stem cells can differentiate into cells representative of the three germ layers, including skin, liver, nerve, muscle, bone, fat, and the like, through in vitro induction culture. Also, they are capable of differentiating into cells characteristic of the three germ layers when transplanted in vivo and capable of surviving and differentiating into organs (e.g., skin, spinal cord, liver, and muscle) when transplanted to the damaged organs via intravenous injection into a living body.

Due to their pluripotency and non-tumorigenicity, the cells or cell populations can be used for treating various degenerative or inherited diseases, while avoiding ethical considerations of human embryo manipulation and tumorigenic risks associated with other stem cells such as ES cells and iPS cells. Furthermore, since the disclosed methods allow one to obtain a large number of pluripotent stem cells, such as MUSE cells, one can also avoid logistical obstacles associated with other types of stem cells.

Thus, this disclosure also provides a pharmaceutical composition comprising the MUSE cells enriched by the methods as described above. In another aspect, this disclosure provides a cell therapy composition for allotransplantation comprising the MUSE cells enriched by the method as described above. The composition may include an appropriate vehicle for delivery of MUSE cells to a subject in need thereof. In some embodiments, the composition may include MUSE cells and a cryo-protectant.

The isolated MUSE cells for treating various conditions, including spinal cord injury, demyelination conditions, traumatic brain injury, and stroke, as well as suppressing unwanted immune responses (e.g., inflammation) and treating disorders of heart, lung, gut, liver, pancreas, muscle, bone marrow, and skin. To that end, one can test the cells for pluripotency first in vitro and then in vivo, and then in uninjured immune-deficient animals, and finally in spinal-injured animals and other models of the central nervous system and other tissue damage.

Accordingly, this disclosure provides a method for regenerating various types of tissue, various organs, and the like. Examples thereof include skin, cerebro-spinal cord, liver, and muscle. The method comprises administering to the subject an effective amount of the MUSE cells enriched by the methods as described above. In some embodiments, the method comprises administering to the subject an effective amount of the first population of cells separated by the above-described methods. In some embodiments, the MUSE cells can be administered directly to or to an area in the vicinity of injured or damaged tissue, organs, and the like, so that the MUSE cells enter the tissue or organ and differentiate into cells unique to the relevant tissue or organ. In this manner, the MUSE cells can contribute to the regeneration or reconstruction of tissue and organs. Also, the systemic administration of the MUSE cells is possible by intravenous administration or the like. In this case, MUSE cells are directed by homing or the like to a damaged tissue or organ, reach and enter the tissue or organ, and then differentiate into cells of the tissue or organ, so as to be able to contribute to tissue or organ regeneration and reconstruction.

Examples of an organ to be regenerated include, but are not limited to, bone marrow, spinal cord, blood, spleen, liver, lungs, bowel, eyes, brain, immune system, circulatory system, bone, connective tissue, muscle, heart, blood vessel, pancreas, central nervous system, peripheral nervous system, kidney, bladder, skin, epithelial appendages, breast-mammary gland, adipose tissue, and mucous membranes of mouth, esophagus, vagina, and anus, for example. Also, examples of diseases to be treated therein include, cancer, cardiovascular disease, metabolic disease, hepatic disease, diabetes mellitus, hepatitis, haemophilia, blood system disease, degenerative or traumatic neurologic disorder such as spinal cord injury, autoimmune disease, genetic defects, connective tissue disease, anemia, infectious disease, graft rejection, ischaemia, inflammation, and damage to skin or muscle.

The cells can be administered to individuals through infusion or injection (for example, intravenous, intrathecal, intramuscular, intraluminal, intratracheal, intraperitoneal, or subcutaneous), orally, transdermally, or other methods known in the art. Also, local administration or systemic administration may be performed herein. Local administration can be performed using a catheter, for example. The dose can be appropriately determined depending on an organ to be regenerated, a tissue type, or a size. Administration may be once every two weeks, once a week, or more often, but frequency may be decreased during a maintenance phase of the disease or disorder.

Cells may be administered with a pharmaceutically acceptable base material. Such base material may be made of a substance with high bio-compatibility, such as collagen or a biodegradable substance. They may be in the form of particles, plates, tubes, vessels, or the like. Cells may be administered after binding thereof to a base material or after causing a base material to contain cells therein.

The present invention encompasses materials for cell transplantation therapy or compositions for cell transplantation therapy, or materials for regeneration medicine or compositions for regeneration medicine, which contain MUSE cells, embryoid body-like cell clusters formed of MUSE cells, and cells or tissue/organs obtained via differentiation from MUSE cells or the above embryoid body-like cell clusters. Such a composition contains a pharmaceutically acceptable buffer, diluent, or the like in addition to MUSE cells, an embryoid body-like cell cluster formed of MUSE cells, or cells or tissue and/or organ obtained through differentiation from MUSE cells or the above embryoid body-like cell cluster.

Both heterologous and autologous cells can be used. In the former case, HLA-matching should be conducted to avoid or minimize host reactions. In the latter case, autologous cells are enriched and purified from a subject and stored for later use. The cells may be cultured in the presence of host or graft T cells ex vivo and re-introduced into the host. This may have the advantage of the host recognizing the cells as self and better providing reduction in T cell activity.

The dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on the recession of pathological signs and clinical and subclinical symptoms of a disease condition or disorder contemplated for treatment with the above-described composition. Dosages and administration regimen can be adjusted depending on the age, sex, physical condition of administered as well as the benefit of the conjugate and side effects in the patient or mammalian subject to be treated and the judgment of the physician, as is appreciated by those skilled in the art. In all of the above-described methods, the cells can be administered to a subject at 1×10⁴ to 1×10¹⁰/time.

Moreover, cells are collected from a patient, MUSE cells are isolated, and then the MUSE cells can be used for various diagnoses. For example, a patient's genes are collected from MUSE cells and then the gene information is obtained, so that precise diagnosis reflecting the information becomes possible. For example, cells of each tissue and/or organ having the same characteristics (e.g., genetic background) as those of a subject can be obtained by causing differentiation of patient's cell-derived MUSE cells. Hence, regarding disease diagnosis, elucidation of pathological conditions, diagnosis for the effects or adverse reactions of drugs, or the like, appropriate diagnosis can be made according to the characteristics of each subject. Specifically, MUSE cells, embryoid body-like cell clusters formed of MUSE cells, and cells or tissue and/or organs obtained through differentiation of MUSE cells or the above embryoid body-like cell clusters can be used as diagnostic materials. For example, the present invention encompasses a method for diagnosing the disease or the like of a subject using MUSE cells isolated from the subject or using tissue or an organ (obtained via differentiation from the MUSE cells) having the same genetic background as that of the subject.

As used herein, the term “subject” refers to a vertebrate, and in some exemplary aspects, a mammal. Such mammals include, but are not limited to, mammals of the order Rodentia, such as mice and rats, and mammals of the order Lagomorpha, such as rabbits, mammals from the order Carnivora, including Felines (cats) and canines (dogs), mammals from the order Artiodactyla, including bovines (cows) and swines (pigs) or of the order Perissodactyla, including Equines (horses), mammals from the order Primates, Ceboids, or Simoids (monkeys) and of the order Anthropoids (humans and apes). In exemplary aspects, the mammal is a mouse. In more exemplary aspects, the mammal is a human.

As used herein, the term “administering” refers to the delivery of cells, such as MUSE cells, by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

As used herein, the term “effective amount” or “therapeutically effective amount” refers to an amount which results in measurable amelioration of at least one symptom or parameter of a specific disorder. A therapeutically effective amount of the above-described cells can be determined by methods known in the art. An effective amount for treating a disorder can be determined by empirical methods known to those of ordinary skill in the art. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of the above-described disorders is within the scope of the invention. Clinically significant attenuation or amelioration means perceptible to the patient and/or to the physician.

Pharmaceutical or cell therapy compositions can be prepared by mixing a therapeutically effective amount of cells and, optionally, other active agents/compounds, with a pharmaceutically acceptable carrier. The carrier refers to a diluent, excipient, or vehicle with which a compound is administered. The carrier can have different forms, depending on the route of administration. The carriers can be sterile liquids, such as water and oils. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. For example, the compositions can be prepared by mixing with conventional pharmaceutical excipients and methods of preparation. Excipients may be mixed with disintegrating agents, solvents, granulating agents, moisturizers, and binders.

The phrase “pharmaceutically acceptable” refers to molecular entities and other ingredients of such compositions that are physiologically tolerable and do not typically produce unwanted reactions when administered to a human. Preferably, the term “pharmaceutically acceptable” means approved by a regulatory agency of the federal or a state government or listed in the U.S. Pharmacopeia or other generally recognized pharmacopeias for use in mammals, and more particularly in humans. Pharmaceutically acceptable salts, esters, amides, and prodrugs refers to those salts (e.g., carboxylate salts, amino acid addition salts), esters, amides, and prodrugs which are, within the scope of sound medical judgment, suitable for use in contact with the tissues of patients without undue toxicity, irritation, allergic response, and the like, commensurate with a reasonable benefit/risk ratio, and effective for their intended use.

### III. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, “about 1” may mean from 0.9-1.1, and “about 4” may mean from 3.6-4.4. Other meanings of “about” may be apparent from the context, such as rounding off, so, for example, “about 1” may also mean from 0.5 to 1.4. The term “about” can refer to a variation of ±5%, ±10%, ±20%, or ±25% of the value specified. For example, “about 50” percent can, in some embodiments, carry a variation from 45 to 55 percent. For integer ranges, the term “about” can include one or two integers greater than and/or less than a recited integer. Unless indicated otherwise herein, the term “about” is intended to include values, e.g., weight percents, proximate to the recited range that are equivalent in terms of the functionality of the individual ingredient, the composition, or the embodiment.

The term “treating” or “treatment” refers to administration of a composition or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### IV. EXAMPLES

### Example 1

This example describes the materials and methods to be used in the subsequent examples.

### Isolation of HUC MSCs

HUC was packed in a bottle filled with the transport medium, which included KH₂PO₄ (0.20 g/L), Na₂HPO₄ (anhydrous, 1.15 g/L), KCl (0.20 g/L), and NaCl (8.00 g/L). The bottle was surrounded by ice to maintain it at 4° C. All the cords were collected with the patients' consent that fulfilled the requirements of the Rutgers University Ethics Committee. The shipment took one day from the patient to the lab. Table I lists the antibodies used in this study.

The isolation of human umbilical cord (HUC) MSCs followed a protocol described as follows. First, the HUC was placed in a 10-cm dish. The HUC was then cut into smaller 1-cm pieces and incised longitudinally. Next, the HUC artery and vein were removed, and the HUC tissues were cleaned, followed by separating Wharton's jelly and cord lining tissues. The tissues were treated with collagenase, and the cells were seeded into cell culture flasks. Briefly, after removal of blood vessels, the mesenchymal tissue was scraped off from the Wharton's jelly with a scalpel and centrifuged at 250×g for 5 minutes at room temperature, and the pellet was washed with serum-free Dulbecco's modified Eagle's medium (DMEM, Gibco, 11330-032). Next, the cells were centrifuged at 250×g for 5 minutes at room temperature and then treated with collagenase Type I solution (SIGMA, SCR103) at a concentration of 2 mg/ml for 16 hours at 37° C. The cells were then washed and treated with 2.5% trypsin (10×) stock solution (THERMOFISHER SCIENTIFIC, 15090046) for 30 minutes at 37° C. with agitation. Finally, the cells were washed and cultured in cell culture medium with 10% fetal bovine serum (FBS, GIBCO 10437-028) in 5% CO₂ in a 37° C. incubator, and the dishes were labeled with information concerning cell passage, name, and date.

### Cell Culture and Passage

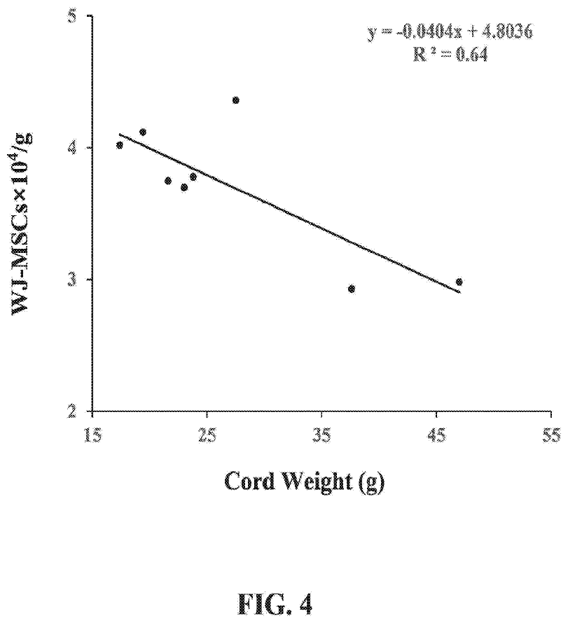

The first seeding of cells from the WJ or CL tissue was named Passage 0 (P0) and subsequent passages were named P1 and P2, etc. The percentages of SSEA3 positive cells in the first three passages were analyzed. The culture medium contained 10% FBS (GIBCO, 10437-028), 2 mM GlutMax (GIBCO, 35050-061), 1% Penicillin-Streptomycin (GIBCO, 15140-122), 1 ng/ml of Human basic Fibroblast Growth Factor (bFGF, PEPROTECH, 100-18B), and the 250 ml bottle was filled with DMEM/F12 (GIBCO, 11330-032). The cells were passaged when they reach 90% confluency (FIG. 4), using the proteolytic enzyme TrypLE™ Express (GIBCO, 12604-013) to release adherent cells from the cell culture dishes, bottles, or plates and replating the cells in additional dishes, bottles, or plates.

### Immunocytochemistry Staining

The cells were transferred at a concentration of 2×10⁴ cells/well to a 24-well plate. Each well had a round coverslip (FISHER SCIENTIFIC, 1254580) at the bottom. After the transfer and appropriate adhesion time, the cells were fixed with 4% paraformaldehyde (0.5 ml/well) and incubated at room temperature (RT) for 10 minutes, followed by washing three times with PBS. The cells were then incubated for 30 minutes with 5% normal goat serum in PBS (without Triton™ X-100, Sigma 234729, for surface markers but with 0.3% Triton™ X-100 for Ki-67 nuclear staining) to block non-specific antibody binding, followed by incubating with primary antibody overnight at 4 degrees. The cells were washed three times with PBS and incubated with secondary antibodies for 30 minutes at RT. As the last step, the cells were incubated in Hoechst 33342 nuclear stain for 10 minutes to label nuclear DNA (THERMOFISHER SCIENTIFIC, 62249).

### Flow Cytometry

The cells (0.3×10⁶ cells) were incubated in a 1.5 ml microcentrifuge tube with primary antibodies. For SSEA3, the primary antibody incubation time was 1 hour at 4° C. and the secondary antibody for 30 minutes. For other antibodies from Miltenyi Biotec, the incubation time was 10 minutes. Before loading, 2.5 μl of 100 μg/ml propidium iodide solution (MILTENYI BIOTEC, 130093233) was added into 500 μl of cell suspension to label cells that may not be viable. The isotype control group was used as controls. The MACSQuant Analyzer 10 Flow Cytometer (MILTENYI BIOTEC), equipped with ten fluorescent channels, was used to perform the cell counts and to produce the graphs.

### Magnetically Activated Cell Sorting (MACS)

Almost all human mesenchymal cells grown on plastic plates express CD105. The MACS procedure positively selects for SSEA3+ cells. About 6×10⁶ cells suspended in 2 ml were loaded into a Magnetic Sorter (MS) column (MILTENYI BIOTEC, 130042201). SSEA3 antibody was added first, following by the addition of anti-rat kappa microbeads MILTENYI BIOTEC, 130047401). The eluted fractions for analyses on MACSquant 10 Flow Cytometer were collected. MS Column should not be loaded with more than 6×10⁶ stained cells suspended in 2 ml. The MS column was washed three times with 1 ml degassed buffer. In the elution step, 2 ml buffer was pipetted into the MS Column. After 3 mins, the plunger was pushed firmly to obtain the magnetically labeled cells. Antibodies used in this study were listed in Table I.

### Doubling Time

To determine cell doubling time (TD), the cells at 5×10³ cells/cm² density were plated, and TD was calculated using the following algorithm (http://www.doubling-time.com):

TD=t×ln 2/(ln Nₜ−ln N₀)

where N₀ is the number of cells inoculated, Nₜ is the number of cells harvested, and t is the culture time in hours. The TD is shown in Table IV for part of the first three passages. The TD of MUSE cells and non-MUSE cells were calculated respectively in every sample.

### Statistical Analysis

SPSS (IBM, R23.0.0.0), AxioVision Rel. 4.8.0 (SP2), and LSM Image Browser (ZEISS Service Pack 2) were used to assess differences among groups using one-way analysis of variance (ANOVA). Post hoc analysis of comparisons among groups was performed using the least significant difference (LSD) test. The results are expressed as the mean±standard deviation (SD), unless otherwise noted. A probability (P-value) of <0.05 was considered significant. An AxioVision Rel. 4.8.0 SP2 and ZEISS LSM Image Browser (Version 4.2.0.121, Zeiss, Wetzlar, Germany) were used to obtain pictures.

### Example 2

Both HUC WJ and CL yielded large numbers of MSCs. Table II showed the number of MSCs and SSEA3+ at Passage 0. The concentrations of MSCs and SSEA3+ cells per gram of tissue had an average of 3.7±0.55×10⁴ WJ-MSCs, 1.89±1.67×10³ WJ-SSEA3+, 3.00±0.80×10⁴ CL-MSCs, and 2.24±2.00×10³ CL-SSEA3+ cells per gram. Heavier cords had more WJ MSCs (R²=0.64, p=0.01<0.05, FIG. 4). The 99WJ group had unusually high 42.37% SSEA+ cells at Passage 0. However, cord weight did not correlate with CL-MSCs/WJ-SSEA3+/CL-SSEA3+. Numbers of WJ-MSCs did not correlate with CL-MSCs or WJ-SSEA3+. Neither between CL-MSCs and CL-SSEA3+.

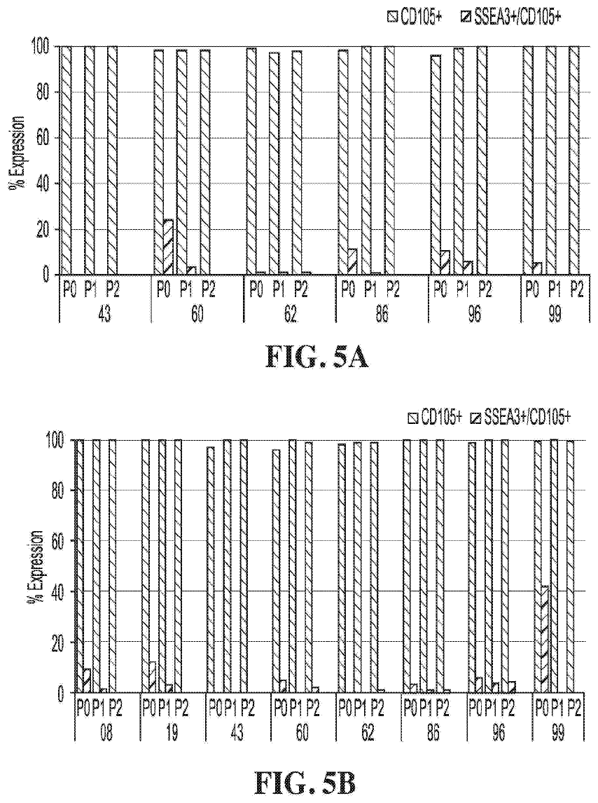

WJ and CL cells were cultured separately, and SSEA3+ percentage over multiple passages were compared (see FIG. 5). In the P0 group, more than 98% of the total cells from both WJ and CL were CD105 positive and even higher in P1 and P2. At P0, the percentages of SSEA3+ cells were 4.97%±4.30% and 5.26%±5.14% in WJ and CL, respectively. However, SSEA3+ percentages dropped sharply between P0 and P1 in the CL group and P0 and P2 in the WJ Group.

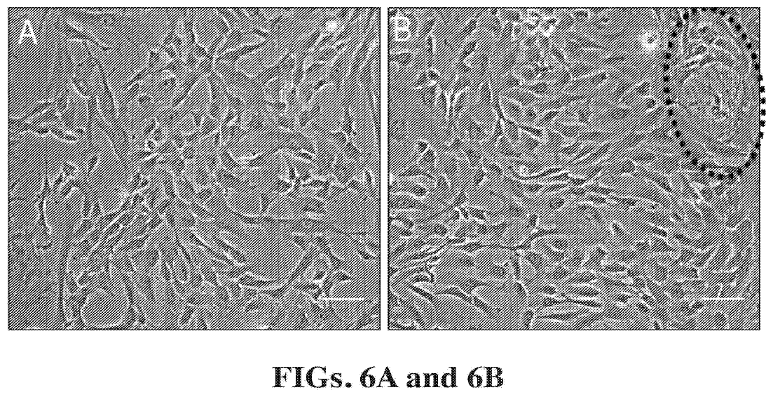

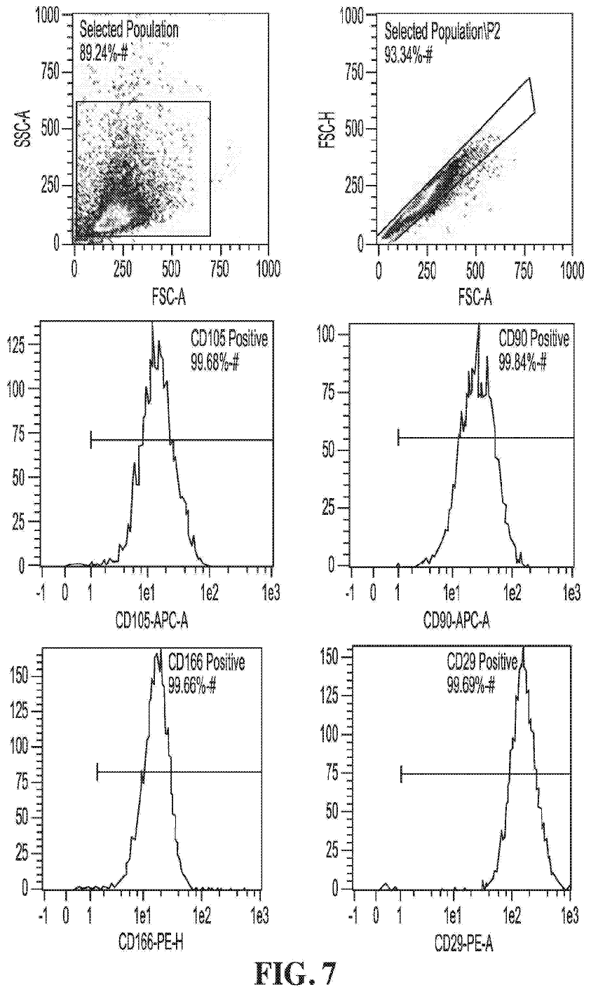

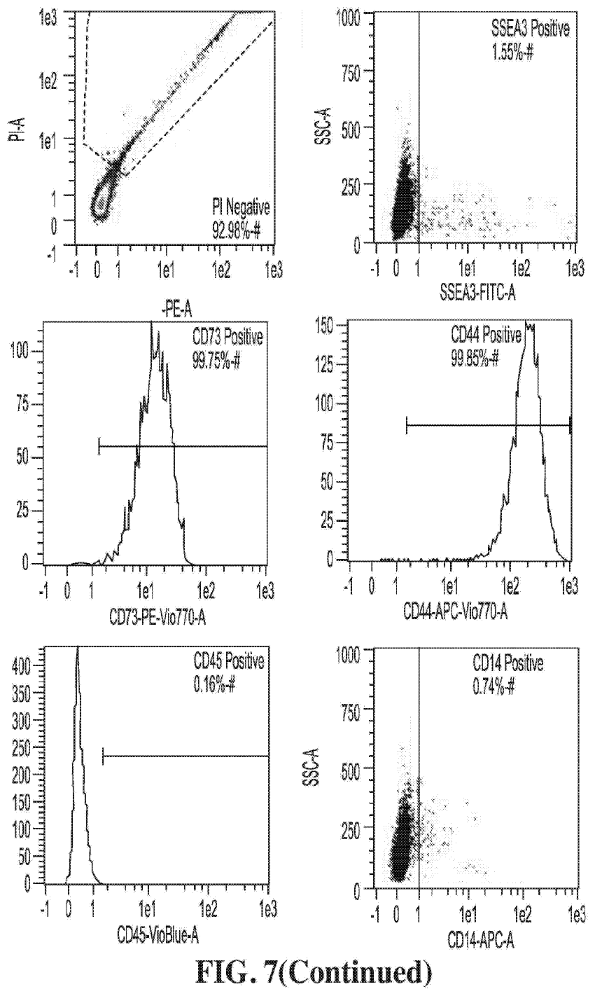

The WJ-MSCs and CL-MSCs had similar morphology (FIG. 6). They were spindle-shaped or triangular with a large oval nucleus in the center of the cell body and one or several nucleoli. In FIG. 6B, the tissue was seeded in the circle as indicated, and the MSCs grew from there. As determined by immunofluorescence, all cells were CD105 positive. SSEA3+ cells often have long, thin processes and trying to make connections with surrounding cells. The flat cell bodies showed irregular shapes but could be as big as 30 μm×100 μm, with large oval nuclei that may be up to 20 μm diameter. Dividing cells had smaller and rounder cell bodies but maintained their typical membrane SSEA3+ staining. In FIG. 7, both WJ and CL-derived MSCs were CD105+, CD90+, CD73+, CD44+, CD166+, and CD29+, but CD45− and CD14−.

Frozen 96WJ P2 cells were cultured, which resulted in an increase of the SSEA3+ cell percentage from 3.91% to 28.27%. Another culture of frozen 96WJ P2 cells resulted in 20.62% SSEA3+ cell percentage, confirming this phenomenon. The freezing process (severe environment) may induce higher MUSE cell percentages. MACS was carried out for each passage from 96WJP2 to 96WJP10.

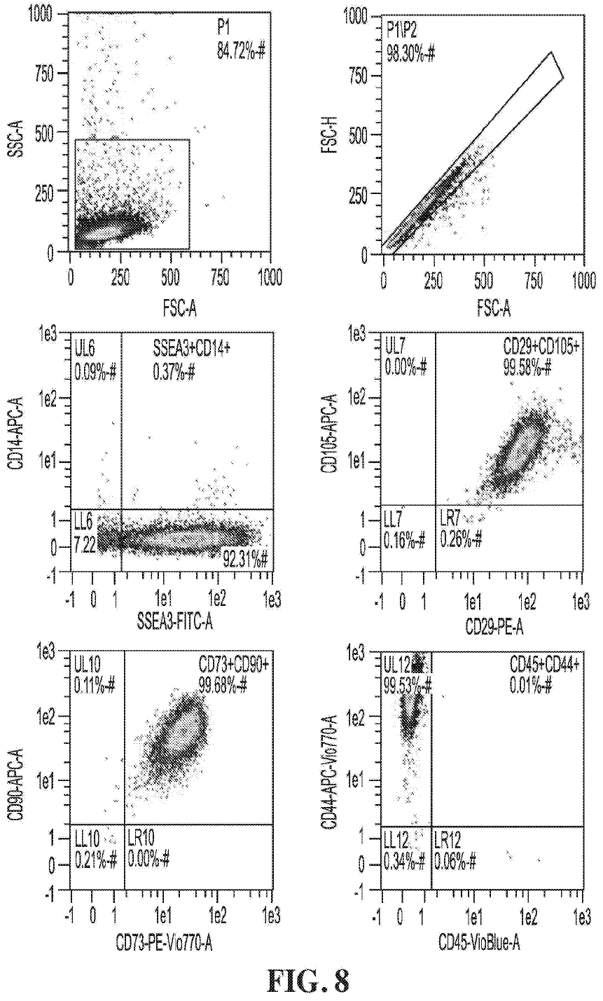

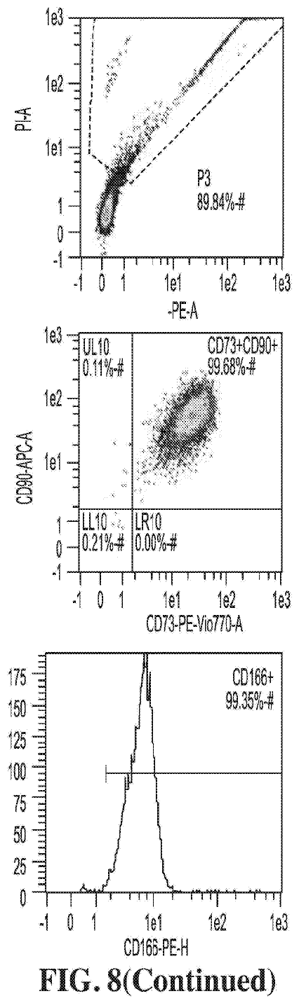

MACS were used to sort 1.23±0.38×10⁵ cells from 1 million MSCs (Table III). Of the sorted population, 91.44%±3.22% were SSEA3+ cells, demonstrating the efficiency of this method. For 96WJP3, the sorting rate was 94.19% and 95.24%, and the average of the others' was 28.31±6.11%, indicating that about 28.31% of the total SSEA3+ cells were sorted from the MSCs population. Further analysis by flow cytometry showed that the sorted population expressed SSEA3 stronger than non-sorted. From the 96WJP8, the percentage of SSEA3+ cells in the MSCs population dropped to 28.10%. The former passages were suggested to be used in the MACS. Further analysis showed that the sorted cells were SSEA3+, CD105+, CD90+, CD29+, CD44+, CD73+ and CD166+, but CD14−, CD45− (FIG. 8).

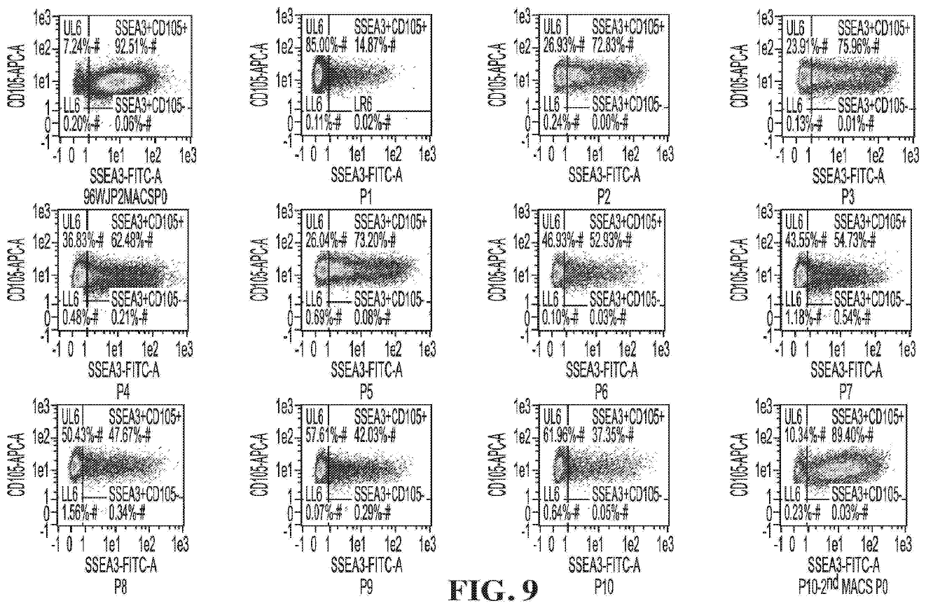

SSEA3+ cell percentages in MACS-sorted 96WJP2 cells during 10 passages were also monitored (see FIG. 9). Right after MACS, 93.8% of the cells were SSEA3+. In the first passage after the MACS, the percentage of SSEA3+ cells decreased to 14.8%, but the number of SSEA3+ cells rebounded, and the cultures maintained 62.48%-75.96% SSEA3+ cells from P2 to P5. The percentage dropped to 42.03%-54.73% from P6 to P9. Even at P10, the cultures had 37.35% SSEA3+ cells. After P10, we re-sorted the cells and achieved an 89.40% SSEA3+ cell culture. The MACS-Culture-reMACS can yield many millions of MUSE cells.

### Example 3

The HUC SSEA3+ and CD105+ cells were transplanted into the spinal cords of two adult Sprague-Dawley rats at 2 weeks after spinal cord injury (SCI) with a 12.5-mm weight drop contusion of the T11 spinal cord. The cells were injected into the dorsal root entry zone of the spinal cords above and below the injury site. The cells survived for 4 weeks after transplantation. The rats were not immunosuppressed. The transplanted cells were stained with an antibody for human nucleus (Stem 101+) but were otherwise negative for Nestin, GFAP, NeuN, NF155, and Iba1. When transplanted into brain and spinal cord, human MUSE cells survive for long times and are not immune-rejected (Uchida H, et al. Stem Cells. 2016; 34(1):160-173; Uchida H, et al. Stroke. 2017; 48(2):428-435).

### Example 4

As demonstrated in this disclosure, the cells respectively isolated from WJ and CL were cultured with collagenase and quantified for the percentages of SSEA3+ cells over three passages. The first passage had 5.0±4.3% and 5.3%±5.1% SSEA3+ cells from WJ and CL, respectively. However, the percentages of SSEA3+ cells fell significantly (p<0.05) between P1 and P2 in the CL group and between P0 and P2 in the WJ Group. Magnetic-activated cell sorting (MACS) markedly enriched SSEA3+ cells to 91.44±3.22%. After the sorted populations were cultured, SSEA3+ percentages ranged from 62.48% to 75.96% between P2 to P5. The percentages of SSEA3+ cells declined to 42%-55% between P6 to P9. Even in P10, the cultures still contained 37% SSEA3+ cells. After P10, the cells were re-sorted and yielded 89% SSEA3+ cultures.

The procedure of enriching for SSEA3+ cells with MACS, followed by expansion in culture, and then re-enriching for SSEA3+ with MACS allows isolation of many millions of SSEA3+ cells in relatively pure cultures. When cultured, the sorted SSEA3+ cells differentiated into embryoid spheres and survived four weeks when transplanted into contused Sprague-Dawley (SD) rat spinal cords. The SSEA3+ cells migrated into the injury area from four injection points around the contusion site and did not produce any tumors. Umbilical cord is an excellent source of fetal MUSE cells, and the disclosed method allows practical and efficient isolation and expansion of relatively pure populations of SSEA3+ MUSE cells that can be matched by human leukocyte antigen (HLA) for transplantation in human trials.

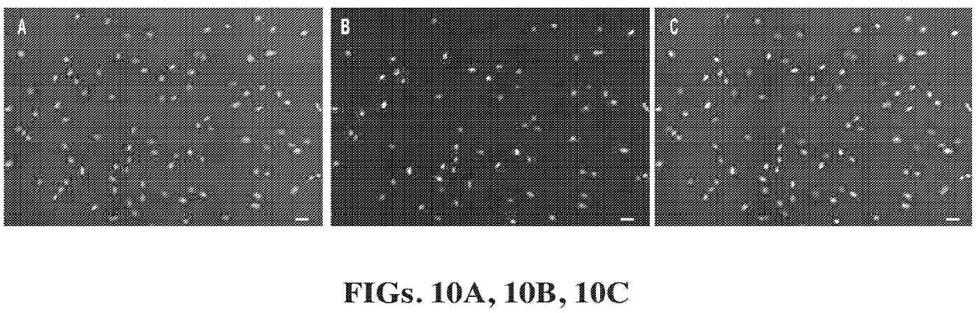

The results showed many SSEA3+ and CD105+ double positive cultured cells in both WJ and CL tissues. SSEA3 is a pluripotent cell surface marker. The SSEA3+ and CD105+ cells are likely to be MUSE cells. However, the percentage of SSEA3+ cells quickly decreased after 2-3 passages, suggesting that non-MUSE (i.e., SSEA3−) cells divided faster than the SSEA3+ population. About 65% of the MSCs (CD105+) were Ki-67 positive (see FIG. 10), indicative of recent proliferation.

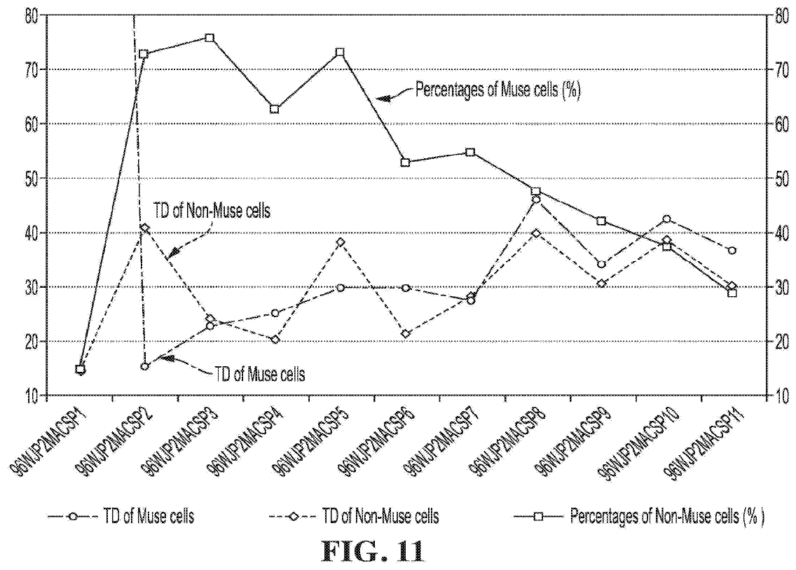

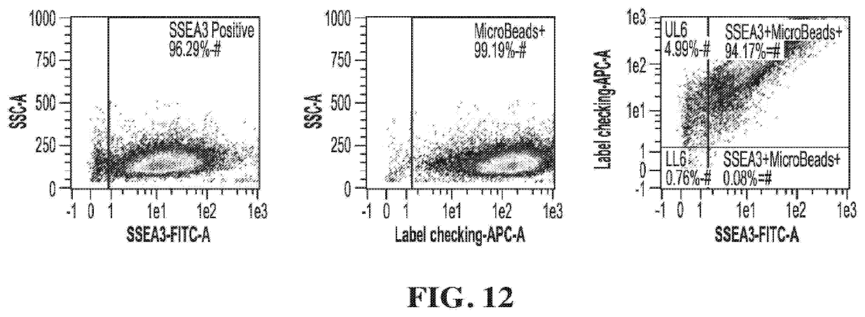

The TD's of Non-MUSE cells were stable, and one-way ANOVA showed no significant differences between CLP1 and CLP2, between WJP1 and WJP2, between CLP1 and WJP1, nor between CLP2 and WJP2. Most of SSEA3+ cells remained in GO with no factors to stimulate them. The minus numbers in Table IV indicate that the cells were not dividing at all. In MACS-sorted SSEA3+ cell populations (FIG. 11), the TD times averaged 30.9±9.2 hours, nearly the same as that of human fibroblasts. The TD times increased with more passages, while the percentages of SSEA3+ cells decreased. From FIG. 11, after the first MACS, P2-P7 appeared to be the best passages to be sorted again for the subsequent transplant experiments. In this study, MACS using the MS Columns efficiently isolated SSEA3+ cells from WJ and CL tissues. Before sorting, <5% of the cells were SSEA3+. After MACS sorting, 91.44±3.22% of cells were SSEA3+. A labeling check reagent was used to make sure the SSEA3 antibody had successfully combined with the Anti-Rat Kappa Microbeads (FIG. 12). Only MUSE cells expressing strong SSEA3 expression (28.31%) were isolated. Two special guidelines for the sorting procedure are provided: (1) Do not load the column with over 6 million cells, and the volume of the cell suspension was 2 ml rather than the suggested 0.5 ml. Otherwise, cells may stick to the column; and (2) In the elution step, pipette 2 ml of the buffer rather than 1 ml and wait 3 minutes before applying the plunger.

Individual SSEA3+ cells showed the typical membrane staining, while previous studies only showed cell clusters with SSEA3 staining. Compared with most human cells within a size range of 2-120 microns, SSEA3+ cells bodies were 25-90 μm similar to macrophages at 20-80 microns, and the nuclei were about 20 μm. Some MUSE cells had very large cell bodies up to 110 μm in length. MUSE cells have many processes that extended toward surrounding cells, while non-MUSE cells do not. Dividing SSEA3+ cells have smaller and round cell bodies of 10 μm while the nucleus is at ˜7 μm.

MACS-sorted SSEA3+ cells cultured in poly-HEMA coated dishes showed small clusters of SSEA3+ cells on Day 2 after plating. Seven days later, many large clusters formed. These clusters were isolated and cultured them in non-poly-HEMA coated wells for 8 hours. Cell clusters attached to the plate and stained for SSEA3. Two studies suggested adding Triton to the blocking solution for immunohistological staining of MUSE cells, but the data showed no SSEA3 signal in 0.3% Triton-treated cell samples (Tian, T., et al. Cellular Reprogramming, 2017. 19(2): p. 116-122). Triton is a detergent that permeabilizes lipid membranes and confirmed that SSEA3 was expressed on the surface of the cells.

The sorted MUSE cells were cultured in a neural precursor cell culture medium: 2% B-27 supplement (50×, THERMO FISHER SCIENTIFIC 17504-044), GlutMax (GIBCO 35050-061, final concentration: 2 mM), bFGF (PEPROTECH 100-18B, final: 30 ng/ml), EGF (PEPROTECH AF-100-15, final: 30 ng/ml), 1% Penicillin-Streptomycin (THERMO FISHER SCIENTIFIC 15140122), and Neurobasal medium (GIBCO 21103049). It took seven days to induce the differentiation into neural precursor cells forming specific spheres. Nestin, NeuN, GFAP, and NF-155 were positive, which indicated the induction was successful, and the neural precursor cells were multipotent.

SSEA3 expression of the transplanted cells could not be determined in the rats because rats were the host species of the SSEA3 antibody. This pilot experiment, however, showed that transplanted human MUSE cells clearly were not immune rejected in the first four weeks after transplantation because the transplanted cells expressed human cytoplasmic and nuclear antigens. Other studies have shown that human MUSE cells survive transplantation and are not immune-rejected in mouse. For example, in a mouse intracerebral hemorrhage model, engrafted human MUSE showed positivity for NeuN (˜57%) and MAP-2 (˜41.6%) at Day 69 (Shimamura, N., et al. Experimental Brain Research, 2017. 235(2): p. 565-572.). Once the MUSE cells differentiated into other cell types, however, they may not survive without immunosuppression.

MUSE cells have immunomodulatory effects (Gimeno, et al., Stem Cell Trans. Medicine, 2017. 6(1): pages 161-173). A similar phenomenon was observed in a study of MSCs (Huang, et al., Circulation. 2010. 122(23): pages 2419-2429) in a rat myocardial infarction model, in which the MHC profile changed, and the immunomodulatory function of allogeneic MSCs associated with differentiation into myocardial cells was lost. Immunosuppressants may be required for longer survival periods of progeny cells of MUSE cells. Migration appears to be guided by S1P-S1PR2, that mediates homing of MUSE cells into a damaged heart for long-lasting tissue repair and functional recovery after acute myocardial infarction (Yamada, et al. Circ. Res. 2018, 122(8): pages 1069-1083)

The finding that SSEA3+ and CD105+ cells grown from HUC survive and migrate after transplantation into injured rat spinal cords without immune-suppression is consistent with the Shimamura's finding that human MUSE cells survived long term when transplanted into the brains of rats after a stroke. The HUC SSEA3/CD105 cells were identified by the antibody Stem 101® (TAKARA, Japan) against a human nuclear protein. This antibody does not recognize mouse, rat, or non-human primate cells. Survival of SSEA3+/CD105+ cells in injured rat spinal cords without immunosuppression is consistent with immune tolerance of human MUSE cells, which express HLA-G.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

TABLE I
Antibodies used in this study

| Antibody | Host & Reaction | Dilution | Manufacture | Catalog # | Usage |
|---|---|---|---|---|---|
| SSEA3 | Rat anti-human | 1:20 | Thermo Fisher | MA1-020 | Immunocytochemistry, |
| | | | | | Flow cytometry, MACS |
| IgMϰ | Rat, Isotype | 1:5 | BD Pharmingen | 550342 | Immunocytochemistry, |
| | | | | | Flow cytometry |
| CD105 | Rabbit anti-human | 1:500 | Thermo Fisher | PAS-12511 | Immunocytochemistry |
| Ki-67 | Rabbit anti-human | 1:500 | Abcam | ab16667 | Immunocytochemistry |
| Stem 101 | Mouse anti-human | 1:500 | Takara Bio, Inc. | Y40400 | Immunocytochemistry |
| Hoechst 33342 | Nucleus staining | 1:1000 | Thermo Fisher | 62249 | Immunocytochemistry |
| FITC-488 conjugated dye | Goat anti-rat IgM | 1:200 | Jackson ImmunoResearch | 112095075 | Immunocytochemistry, |
| | | | | | Flow cytometry |
| Alexa Fluor 555 conjugated dye | Goat anti-mouse | 1:500 | Thermo Fisher | A-21422 | Immunocytochemistry |
| Alexa Fluor 555 conjugated dye | Goat Anti-Rabbit | 1:1000 | Abcam | ab150086 | Immunocytochemistry |
| CD105-APC | Anti-human | 1:11 | Miltenyi Biotec | 130094926 | Flow cytometry |
| CD90-APC | Anti-human | 1:11 | Miltenyi Biotec | 130095402 | Flow cytometry |
| IgG1-APC | Mouse, Isotype | 1:11 | Miltenyi Biotec | 130095902 | Flow cytometry |
| CD44-APC-Vio770 | Anti-human | 1:11 | Miltenyi Biotec | 130099149 | Flow cytometry |
| IgG1-APC-Vio770 | Mouse, Isotype | 1:11 | Miltenyi Biotec | 130096653 | Flow cytometry |
| CD29-PE | Anti-human | 1:11 | Miltenyi Biotec | 130101273 | Flow cytometry |
| IgG1-PE | Mouse, Isotype | 1:11 | Miltenyi Biotec | 130095900 | Flow cytometry |
| CD73-PE-Vio770 | Anti-human | 1:11 | Miltenyi Biotec | 130104192 | Flow cytometry |
| IgG1-PE-Vio770 | Mouse, Isotype | 1:11 | Miltenyi Biotec | 130096654 | Flow cytometry |
| CD45-VioBlue | Anti-human | 1:11 | Miltenyi Biotec | 130092880 | Flow cytometry |
| IgG2a-VioBlue | Mouse, Isotype | 1:11 | Miltenyi Biotec | 130094671 | Flow cytometry |

TABLE II
The records of the eight HUCs showed the numbers of MSCs and SSEA3+ at Passage 0

| | Cord | | Net | | | HUC-MSCs/g | | | Total SSEA3+ |
|---|---|---|---|---|---|---|---|---|---|
| | Number/ | Collection | Weight | Two | HUC-MSCs | cord | SSEA3+ cells | Total SSEA3+ | cells/g cord |
| No. | Briefly | Date | (g) | Groups | (×10⁶) | (×10⁴ cells/g) | in MSCs (%) | cells (×10³) | (×10³/g) |
| 1 | C-3561762/62 | Jan. 06, 2017 | 21.6 | WJ | 0.81 | 3.75 | 0.40 | 3.24 | 0.15 |
| | | | | CL | 0.70 | 3.24 | 0.30 | 2.10 | 0.10 |
| 2 | C-3561760/60 | Jan. 10, 2017 | 27.5 | WJ | 1.20 | 4.36 | 5.00 | 60.0 | 2.18 |
| | | | | CL | 0.57 | 2.07 | 24.00 | 136.8 | 4.97 |
| 3 | C-3561819/19 | Jan. 11, 2017 | 17.4 | WJ* | 0.70 | 4.02 | 11.27 | 78.89 | 4.53 |
| 4 | C-3561843/43 | Jan. 16, 2017 | 37.6 | WJ | 1.10 | 2.93 | 0.01 | 0.11 | 0.00 |
| | | | | CL | 1.10 | 2.93 | 0.19 | 0.21 | 0.01 |
| 5 | C-3561808/08 | Jan. 19, 2017 | 19.4 | WJ* | 0.80 | 4.12 | 8.38 | 67.04 | 3.46 |
| 6 | C-3561886/86 | Jan. 19, 2017 | 23.0 | WJ | 0.85 | 3.70 | 3.08 | 26.18 | 1.14 |
| | | | | CL | 0.50 | 2.17 | 10.90 | 54.5 | 2.37 |
| 7 | C-3561896/96 | Jan. 27, 2017 | 47.0 | WJ | 1.40 | 2.98 | 5.93 | 83.02 | 1.77 |
| | | | | CL | 1.60 | 3.40 | 11.61 | 185.76 | 3.95 |
| 8 | C-3561899/99 | Jan. 30, 2017 | 23.8 | WJ | 0.90 | 3.78 | 42.37 | 381.33 | 16.02 |
| | | | | CL | 1.00 | 4.20 | 4.88 | 48.8 | 2.05 |

*The MSCs were not successfully derived from CL tissues of No. 19 and 08

Table III
MACS Performances from 96WJP2 to 96WJP10

| | Total live cells | Percentages | Number | Number | Percentages of | Number of | Sorting |
|---|---|---|---|---|---|---|---|
| | before MACS | of MUSE cells | of MUSE cells | of sorted cells | MUSE in sorted | sorted MUSE | rate |
| Passage | (×10⁶) | before MACS (%) | before MACS (×10⁵) | (×10⁵) | population | cells (×10⁵) | (%)* |
| 96WJP2 | 5.24 | 28.27 | 14.81 | 3.60 | 93.84 | 3.38 | 22.80 |
| 96WJP3 | 6.00 | 14.80 | 8.88 | 9.00 | 92.94 | 8.36 | 94.19 |
| 96WJP3 | 6.00 | 14.80 | 8.88 | 9.10 | 92.94 | 8.46 | 95.24 |
| 96WJP4 | 5.00 | 48.64 | 24.32 | 7.50 | 92.56 | 6.94 | 28.54 |
| 96WJP4 | 5.20 | 41.12 | 21.38 | 8.00 | 89.85 | 7.19 | 33.61 |
| 96WJP5 | 6.00 | 59.42 | 35.65 | 9.40 | 88.35 | 8.30 | 23.29 |
| 96WJP5 | 4.50 | 40.45 | 18.20 | 6.00 | 93.36 | 5.60 | 30.77 |
| 96WJP6 | 6.00 | 43.95 | 26.37 | 10.00 | 96.29 | 9.63 | 36.51 |
| 96WJP7 | 5.70 | 47.87 | 27.28 | 7.70 | 94.45 | 7.27 | 26.65 |
| 96WJP8 | 6.00 | 28.10 | 16.86 | 4.50 | 91.02 | 4.10 | 24.29 |
| 96WJP8 | 6.00 | 28.10 | 16.86 | 3.60 | 91.02 | 3.28 | 19.43 |
| 96WJP9 | 6.20 | 25.66 | 15.90 | 5.00 | 84.22 | 4.21 | 26.46 |
| 96WJP10 | 2.60 | 25.98 | 6.75 | 3.00 | 87.91 | 2.64 | 39.04 |

Sorting rate (%) = Number of sorted MUSE cells/Number of MUSE cells before MACS *100%

TABLE IV
WJ and CL cell doubling times of the first three passages (hours)

| | TD of | TD of | | TD of | TD of |
|---|---|---|---|---|---|
| | Non-MUSE | MUSE | | Non-MUSE | MUSE |
| | cells | cells | | cells | cells |
| Passages | (hrs) | (hrs) | Passages | (hrs) | (hrs) |
| 60CLP1 | 30.1 | −592.4 | 60CLP2 | 54.8 | −25.5 |
| 62CLP1 | 48.6 | 41.5 | 62CLP2 | 48.0 | 45.7 |
| 43CLP1 | 42.5 | 239.3 | 43CLP2 | 54.0 | 86.4 |
| 99CLP1 | 37.6 | −47.5 | 99CLP2 | 48.0 | 40.2 |
| mean | 39.7 | NA | mean | 51.2 | NA |
| SD | 7.8 | NA | SD | 3.7 | NA |
| 60WJP1 | 61.8 | −31.6 | 60WJP2 | 50.6 | 11.5 |
| 62WJP1 | 51.0 | −156.6 | 62WJP2 | 41.5 | 14.0 |
| 86WJP1 | 50.5 | 108.4 | 86WJP2 | 61.5 | 56.1 |
| 96WJP1 | 44.1 | 52.9 | 08WJP2 | 90.4 | −71.7 |
| 99WJP1 | 44.5 | −78.8 | 19WJP2 | 67.5 | −54.9 |
| mean | 50.4 | NA | Mean | 62.3 | NA |
| SD | 7.2 | NA | SD | 18.6 | NA |

What is claimed is:

1. A method of enriching multi-lineage stress enduring (MUSE) cells, comprising:
(i) providing a cell or tissue source of MUSE cells;
(ii) separating a first population of cells from the cell or tissue source of MUSE cells, wherein the first population of cells is separated by selecting for SSEA3+ cells and comprises SSEA3+ MUSE cells;
(iii) culturing at least a sub-population of the first population of cells in a culture medium;
(iv) repeating step (iii) at least 1-10 passages; and
(v) separating from resulting cultured cells a population of enriched MUSE cells by selecting for SSEA3+ cells, whereby the population of enriched MUSE cells comprises about or greater than 80% of SSEA3+ MUSE cells.

2. The method of claim 1, further comprising:
separating from the cell or tissue source of MUSE cells a second population of cells and a third population of cells, wherein the second population of cells is separated by selecting for CD4+ and CD8+ cells before or after the first population of cells are separated from the cell or tissue source of MUSE cells, and the third population of cells is recovered after the first population of cells and the second population of cells are separated from the cell or tissue source of MUSE cells.

3. The method of claim 1, wherein the culture medium comprises basic fibroblast growth factor (bFGF).

4. The method of claim 2, wherein the second population of cells comprises T- and natural killer (NK) lymphocytes.

5. The method of claim 2, wherein the third population of cells comprises CD14+ monocytes, CD34+ endothelial progenitor cells, or CD133+ pluripotent cells

6. The method of claim 1, wherein the cell or tissue source of MUSE cells is obtained from a tissue of an animal.

7. The method of claim 6, wherein the tissue is selected from the group consisting of umbilical cord blood, umbilical cord, umbilical cord stroma cells (Wharton's jelly), amniotic membranes, placenta, umbilical cord lining, menstrual blood, peripheral blood, bone marrow, skin, and adipose.

8. The method of claim 6, wherein the tissue is umbilical cord blood.

9. The method of claim 1, wherein the cell or tissue source of MUSE cells comprises mesenchymal cells.

10. The method of claim 1, wherein the cell or tissue source of MUSE cells comprises mononuclear cells.

11. The method of claim 6, wherein the animal is a mammal.

12. The method of claim 11, wherein the mammal is a human.

13. The method of claim 1, wherein separating the first population of cells is performed using an immunoaffinity-based reagent comprising an SSEA3 antibody.

14. The method of claim 2, wherein separating the second population of cells is performed using an immunoaffinity-based reagent comprising CD4 and CD8 antibodies.

15. The method of claim 13, wherein the SSEA3 antibody is a monoclonal antibody.

16. The method of claim 15, wherein the SSEA3 antibody is a mouse or rat monoclonal IgG or IgM antibody.

17. The method of claim 13, wherein the SSEA3 antibody is conjugated to magnetic particles.

18. The method of claim 14, wherein the CD4 and CD8 antibodies are monoclonal antibodies.

19. A pharmaceutical composition, comprising the MUSE cells enriched by the method of claim 1.

20. A cell therapy composition for allotransplantation, comprising the MUSE cells enriched by the method of claim 1.

21. A method for regenerating a tissue in a subject, comprising administering to the subject an effective amount of the MUSE cells enriched by the method of claim 1.
